(19) United States
(12) Patent Application Publication
(10) Pub. No.: US 2019/0041393 A1
(43) Pub. Date: Feb. 7, 2019

(54) PEPTIDE ASSIGNMENT METHOD AND PEPTIDE ASSIGNMENT SYSTEM

(71) Applicant: Shimadzu Corporation, Kyoto-shi, Kyoto (JP)

(72) Inventors: Masaki MURASE, Kyoto-shi (JP); Koichi TANAKA, Kyoto-shi (JP)

(73) Assignee: Shimadzu Corporation, Kyoto-shi, Kyoto (JP)

(21) Appl. No.: 15/759,659

(22) PCT Filed: Sep. 13, 2016
(86) PCT No.: PCT/JP2016/076963

§ 371 Date: Mar. 13, 2018

(30) Foreign Application Priority Data
Sep. 14, 2015 (JP) 2015-181031

(51) Int. Cl. G01N 33/68

(52) U.S. Cl. CPC G01N 33/6848

(57) ABSTRACT

Based on an endogenous peptide whose peptide sequence is known among endogenous peptides produced in vivo and on a full-length sequence of a precursor protein of the endogenous peptide, a database creation unit 11 generates a peptide sequence as a target peptide sequence, the peptide sequence containing one or more residues of a partial sequence of the endogenous peptide, thereby creating a target peptide sequence database 111 including a plurality of the target peptide sequences. The mass spectrometry unit 12 performs mass spectrometry on a peptide sample. A peptide assignment unit 14 determines a peptide sequence of an endogenous peptide contained in the peptide sample based on the plurality of target peptide sequences generated by the database creation unit 11 and on a mass spectrum obtained by the mass spectrometry unit 12.

### TECHNICAL FIELD

The present invention relates to a peptide assignment method and a peptide assignment system for determining a peptide sequence of an endogenous peptide produced (generated) in vivo.

### BACKGROUND ART

As typical peptide assignment methods targeted to proteins, there have been known: a method using a database search (for example, see Non-Patent Document 1 below); and a method using de novo sequencing (see, for example, Non-Patent Document 2 below).

For the method using the database search, for example, a database search method such as Mascot provided by Matrix Science Ltd. is used (see, for example, Non-Patent Document 1 below). Specifically, all combinations of peptide fragments, which are assumed from protein amino acid sequences contained in a protein database, are obtained by in silico digestion. Then, molecular weights of the peptide fragments in the obtained combinations are collated with an MS² precursor ion mass. Theoretical product ion masses are calculated for the peptide fragments whose molecular weights coincide with the MS² precursor ion mass within a predetermined mass tolerance range. The calculated theoretical product ion masses are collated with MS² measurement data, and a peptide having a high degree of matching is searched for.

Meanwhile, in the de novo sequencing, amino acid sequences are read from measurement data without using the database. Specifically, groups of peptide fragments from which amino acid residues are removed from ends of peptides one by one by some method are generated, and the amino acid sequences are read from mass differences in ion peaks derived from the groups of the peptide fragments. As a typical implementation, software called PEAKS is widely known (for example, see Non-Patent Document 2 below).

In the analysis targeted to proteins, whichever of the peptide assignment methods as described above may be used, proteins are frequently fragmented into peptides using proteases in order to reduce difficulty of the analysis, and then the analysis is performed. As described above, the proteins are fragmented and reduced in size, whereby ionization thereof during mass spectrometry is promoted, and analytical sensitivity is improved.

In this case, selection of the proteases is also important in data analysis. For example, in the database search using the protein database, such a protease that cleaves the proteins at specific sites (specific sequences) is selected, whereby a search space can be reduced as compared with a method of randomly cleaving the proteins. As a result, a number of peptide identifications can be increased while keeping a search time and misidentification to a practical level. Moreover, in the de novo sequencing, the protease can be selected so that product ions are generated, the product ions facilitating sequencing of an amino acid distribution within each peptide. For example, when trypsin is selected as such a protease, the sequencing is facilitated since y/b series ions are specifically generated when the ions are cleaved by collision induced dissociation (CID).

### PRIOR ART DOCUMENTS

### Non-Patent Documents

Non-Patent Document 1: “Matrix Science-Mascot-MS/MS-Ions Search”, [online], UK Matrix Science Ltd., [searched on Oct. 22nd, 2012], Internet <URL: http://www.matrixscience.com/cgi/search_form.pl?FORMVER=2&SEARCH=MIS>

Non-Patent Document 2: Bin Ma, et al.: An effective algorithm for the peptide De Novo sequencing from MS/MS spectrum. Symp. Comb. Pattern Matching 2003, 266-278

### SUMMARY OF THE INVENTION

### Problems to be Solved by the Invention

Endogenous peptides are peptides produced in vivo, and are transported as molecules, which relate to information transmission and function control in a body, through body fluid such as blood. Some are released as metabolic products into urine. By analyzing a structure of the endogenous peptides, it is possible to obtain information useful for development of new drugs, diagnosis of diseases, and the like. However, it has been difficult to use the conventional peptide assignment method as described above for the analysis of the endogenous peptides.

Specifically, the endogenous peptides are produced by cleaving proteins by in-vivo processing machineries and metabolic machineries. From one protein, a large number of cleaved peptide variants are produced, and some of these cleaved peptides may include cleaved peptides which share partial sequences and cleaved peptides which do not share the partial sequences. In proteome analysis, proteins are cleaved at specific sites using proteases, so that it is possible to assign the entire proteins by determining a sequence of partial cleaved peptides with a high detection amount (ionization efficiency).

However, the endogenous peptides are produced in vivo by various processing machineries, so that a production amount of peptides derived from the same protein varies. Therefore, for peptides which are low in production amount and ionization efficiency and are difficult to detect, a higher sensitive assignment technique is required. Moreover, except for endogenous peptides whose processing machinery is known, processing sites of the endogenous peptides from proteins are not specified in advance. Therefore, in order to analyze the structure of the endogenous peptide by the database search using a protein database, which is a conventional method, it is necessary to perform a search assuming cleaved peptides produced by processing at every site. Accordingly, a search space increases significantly. Such a significant increase in the search space not only results in an increase in a search time but also in a problem that the number of peptide identifications decreases, that is, identification sensitivity decreases.

In addition, since the endogenous peptides have diverse spatial distributions of amino acids within the peptides, product ion generation patterns are diverse and complicated. Therefore, the analysis by the de novo sequencing also becomes difficult unlike the case where an amino acid distribution of the cleaved peptides is designed to be homogeneous by introduction of proteases like the proteome analysis.

The present invention has been made in view of the above circumstances. It is an object of the present invention to provide a peptide assignment method and a peptide assignment system which are capable of determining peptide sequences of more endogenous peptides with high sensitivity.

### Means for Solving the Problems

A peptide assignment method according to the present invention includes: a database creation step; a mass spectrometry step; and a peptide assignment step. In the database creation step, based on an endogenous peptide whose peptide sequence is known among endogenous peptides produced in vivo and on a full-length sequence of a precursor protein of the endogenous peptide, a peptide sequence containing one or more residues of a partial sequence of the endogenous peptide is generated as a target peptide sequence, whereby a target peptide sequence database including a plurality of the target peptide sequences is created. In the mass spectrometry step, mass spectrometry is performed on a peptide sample. In the peptide assignment step, a peptide sequence of an endogenous peptide contained in the peptide sample is determined based on the plurality of target peptide sequences generated by the database creation step and on a mass spectrum obtained by the mass spectrometry step.

With such a configuration, based on the endogenous peptide whose peptide sequence is known and on the full-length sequence of the precursor protein of the endogenous peptide, the database of the peptide sequences, each containing one or more residues of the partial sequence of the endogenous peptide, is created. The peptide sequence (target peptide sequence) in which the partial sequence is partially common to the endogenous peptide whose peptide sequence is known may be left as a peptide sequence of an unknown endogenous peptide in a mass spectrum that is not assigned by a conventional method.

Therefore, if the database (target peptide sequence database) of the target peptide sequences is generated, then the increase in the search space can be effectively prevented. Then, based on the target peptide sequence database and the mass spectrum obtained by the mass spectrometry of the peptide sample, the peptide sequence of the target peptide contained in the peptide sample is preferentially searched for, whereby more peptide sequences of the endogenous peptides can be determined with high sensitivity.

A peptide assignment system according to the present invention includes: a database creation unit; a mass spectrometry unit; and a peptide assignment unit. Based on an endogenous peptide whose peptide sequence is known among endogenous peptides produced in vivo and on a full-length sequence of a precursor protein of the endogenous peptide, the database creation unit generates a peptide sequence as a target peptide sequence, the peptide sequence containing one or more residues of a partial sequence of the endogenous peptide, thereby creating a target peptide sequence database including a plurality of the target peptide sequences. The mass spectrometry unit performs mass spectrometry on a peptide sample. The peptide assignment unit determines a peptide sequence of an endogenous peptide contained in the peptide sample based on the plurality of target peptide sequences generated by the database creation unit and on a mass spectrum obtained by the mass spectrometry unit.

### Effects of the Invention

In accordance with the present invention, the target peptide sequence database is created, whereby the increase of the search space can be prevented effectively, and more peptide sequences of the endogenous peptides can be determined with high sensitivity based on the target peptide sequence database and the mass spectrum obtained by the mass spectrometry of the peptide sample.

### BRIEF DESCRIPTION OF THE DRAWINGS

FIG. 1 is a block diagram showing a configuration example of a peptide assignment system according to a first embodiment of the present invention.

FIG. 2 is a diagram for describing an aspect when a target peptide sequence is generated by a database creation unit.

FIG. 3 is a flowchart showing processing by a database creation unit.

FIG. 4 is a flowchart showing processing by a mass spectrometry unit and a peak list creation unit.

FIG. 5 is a flowchart showing processing by a peptide assignment unit.

FIG. 6 is a table showing results of actually analyzing MS² spectra obtained from urine samples.

FIG. 7 is a block diagram showing a configuration example of a peptide assignment system according to a second embodiment of the present invention.

### MODE FOR CARRYING OUT THE INVENTION

### First Embodiment

### 1. CONFIGURATION OF PEPTIDE ASSIGNMENT SYSTEM ACCORDING TO FIRST EMBODIMENT

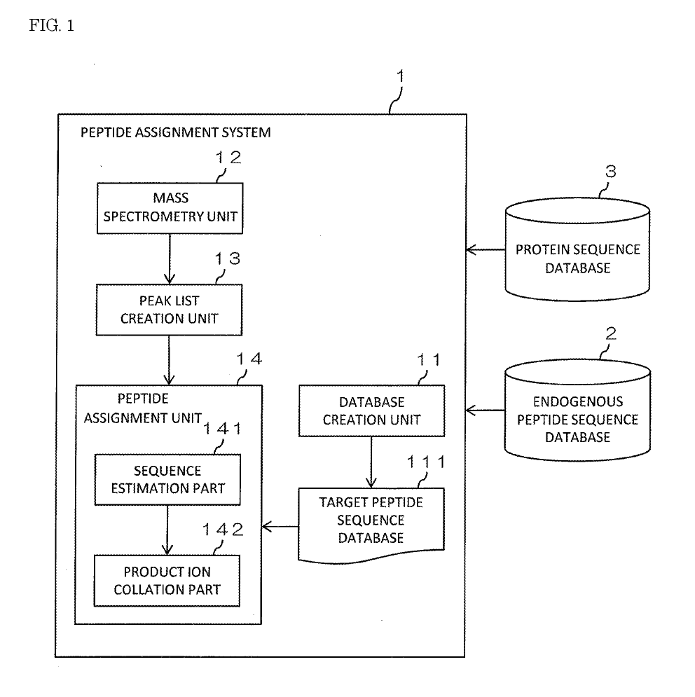

FIG. 1 is a block diagram showing a configuration example of a peptide assignment system 1 according to a first embodiment of the present invention.

The peptide assignment system 1 is for determining a peptide sequence of an endogenous peptide produced in vivo from samples (peptide samples) to be analyzed. The peptide assignment system 1 includes a database creation unit 11, a mass spectrometry unit 12, a peak list creation unit 13, a peptide assignment unit 14, and the like. At least a part of each of the units 11 to 14 is composed of an information processing apparatus including a central processing unit (CPU).

The peptide assignment system 1 uses a plurality of peptide sequences stored in an endogenous peptide sequence database 2 to determine the peptide sequence of the endogenous peptide in the peptide samples. In the endogenous peptide sequence database 2, with regard to a plurality of endogenous peptides whose peptide sequences are known, peptide sequences of those endogenous peptides are stored. Herein, the phrase “peptide sequences are known” is a concept including a case where the peptide sequences are contained in a published sequence database of endogenous peptides or in document information thereof, and a case where the peptide sequences are assigned with high reliability by a conventional analysis method (including manual analysis).

As the published sequence database of the endogenous peptides and the document information thereof, for example, there are known: Mosaiques DB (http://mosaiques-diagnostics.de/diapatpcms/mosaiquescms/front_content.php?idcat=257, Siwy et al., “Human urinary peptide database for multiple disease biomarker discovery”, Proteomics Clin. Appl., 2011, 5, 367-374), which is a sequence database of endogenous peptides contained in urine; an undatabased document (Smith, et al., “Deciphering the peptidome of urine from ovarian cancer patients and healthy controls”, Clin. Proteomics, 2014, 11 (1): 23); and the like.

Based on the peptide sequences of the plurality of endogenous peptides stored in the endogenous peptide sequence database 2 and on full-length sequences of precursor proteins of the endogenous peptides stored in a protein sequence database 3, the database creation unit 11 generates peptide sequences different from these endogenous peptides as target peptide sequences. In this way, the database creation unit 11 creates a target peptide sequence database 111 including a plurality of target peptide sequences (database creating step). The protein sequence database 3 is a full-length sequence database of proteins referred to when unregistered sequences are extended in the endogenous peptide sequence database 2. The target peptide sequences generated at this time are peptide sequences each containing one or more residues of a partial sequence of the endogenous peptide whose peptide sequence is stored in the endogenous peptide sequence database 2. That is, the target peptide sequences generated by the database creation unit 11 have partial sequences (or all sequences) in common with endogenous peptides having known peptide sequences.

The mass spectrometry unit 12 performs mass spectrometry on such a peptide sample (mass spectrometry step). A method of the mass spectrometry by the mass spectrometry unit 12 is not limited, but for example, a method using an ion trap time-of ffight mass spectrometer (IT-TOF MS) can be adopted. When this method is used, the mass spectrometry is performed on the peptide sample using the IT-TOF MS equipped with, for example, an ionization unit, an ion trap and a time of ffight mass spectrometer (TOF MS) (neither is shown).

Specifically, the peptide sample is ionized in the ionization unit, and the obtained ions are trapped by the ion trap. As the ion trap, for example, a three-dimensional quadrupole type can be used, but the present invention is not limited to this. A part of the trapped ions is selectively left in the ion trap, and the ions are cleaved by collision induced dissociation (CID). The cleaved ions are sent from the ion trap to the TOF MS (time of ffight mass spectrometry).

In the TOF MS, ions which have flown in a flight space are detected by a detector. Specifically, the ions accelerated by an electric field formed in the flight space are temporally separated according to m/z (mass-to-charge ratio) while flying in the ffight space, and are sequentially detected by the detector. As a result, a relationship between m/z and detection intensity at the detector is measured as a mass spectrum, and mass spectrometry is achieved. However, not only the IT-TOF MS but also a hybrid mass spectrometer such as a quadrupole time-of-ffight mass spectrometer (Q-TOF MS) and a quadrupole ion trap mass spectrometer (Qq-IT MS) may be used for mass spectrometry as well as a tandem time-of-ffight mass spectrometer (Tandem TOF (TOF-TOF) MS) without an ion trap. Moreover, the cleavage method of ions is not limited to the CID, and other cleavage methods such as electron transfer dissociation (ETD) and electron capture dissociation (ECD) may be used.

In the mass spectrometry using the IT-TOF MS, MSⁿ analysis (n is an integer of 2 or more) is performed by repeatedly performing a series of operations of cleaving ions in the ion trap and performing mass analysis by the TOF MS, whereby an MSⁿ spectrum as a mass spectrum can be measured.

Based on the MSⁿ spectrum obtained by the mass spectrometry unit 12, the peak list creation unit 13 creates a peak list (MSⁿ peak list) in which peaks included in the MSⁿ spectrum are extracted.

Based on the plurality of target peptide sequences stored in the target peptide sequence database 111 and on the peak list created by the peak list creation unit 13, the peptide assignment unit 14 determines the peptide sequence of the endogenous peptide contained in the peptide sample (peptide assignment step). The peptide assignment unit 14 includes functional units such as a sequence estimation part 141 and a product ion collation part 142, for example, by the CPU executing a program.

For example, for MS² precursor ions in an MS¹ peak list, the sequence estimation part 141 searches the target peptide sequence database 111 for peptide sequences which match an MS² precursor ion mass within a predetermined mass tolerance range. The peptide sequences searched by the sequence estimation part 141 become candidates for the peptide sequence (peptide sequence candidate) of the endogenous peptide contained in the peptide sample.

The product ion collation part 142 scores the peptide sequence candidates obtained by the sequence estimation part 141. When the peptide sequence candidates are obtained sufficiently in number, a statistically significant peptide sequence candidate is obtained from a distribution of score of the respective peptide sequence candidates, and the peptide sequence concerned can be determined as the peptide sequence of the endogenous peptide contained in the peptide sample.

### 2. PROCESSING BY DATABASE CREATION UNIT

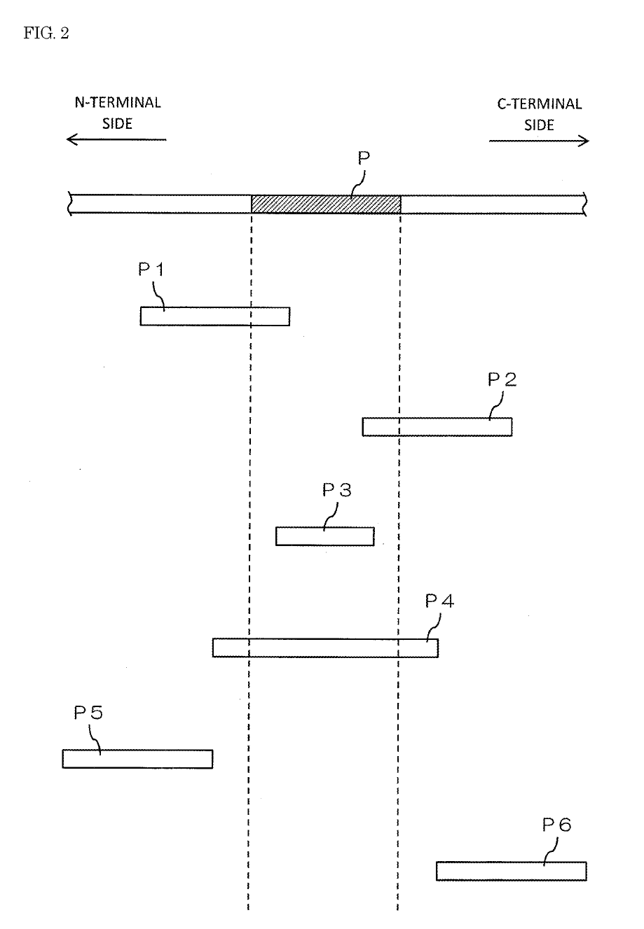

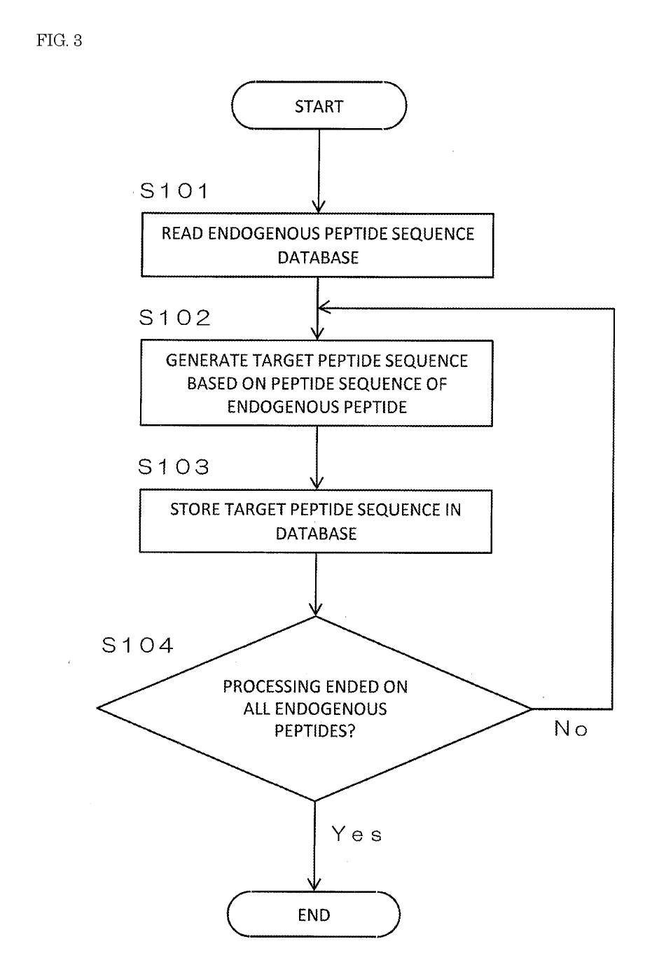

FIG. 2 is a diagram for describing an aspect when the target peptide sequence is generated by the database creation unit 11. FIG. 3 is a flowchart showing processing by the database creation unit 11.

In an example of FIG. 2, a description will be given of a case where an endogenous peptide P having the known peptide sequences are included in a protein (assignment protein) having a known full-length sequence. That is, in this example, it is assumed that a peptide sequence of the endogenous peptide P is stored in the endogenous peptide sequence database 2. It is preferable that the endogenous peptide P in which the peptide sequence is stored in the endogenous peptide sequence database 2 be assigned to the protein, and be given the full-length sequence of this protein and a sequence starting residue and sequence ending residue of the endogenous peptide P in the full-length sequence.

In this case, the database creation unit 11 reads the endogenous peptide sequence database 2 (Step S101), and generates the peptide sequence of the target peptide (that is, the target peptide sequence) based on the read peptide sequence of each endogenous peptide P (Step S102). Specifically, the database creation unit 11 generates the target peptide sequence by extending and contracting the peptide sequence while leaving a part (partial sequence) of the peptide sequence of the endogenous peptide P at least one residue. At this time, the database creation unit 11 extends and contracts the peptide sequence while referring to the full-length sequence of the assignment protein containing the endogenous peptide P.

The database creation unit 11 stores the generated target peptide sequence in the target peptide sequence database 111 (Step S103). The processing in Steps S102 and S103 is performed on all the endogenous peptides P stored in the endogenous peptide sequence database 2. When the processing on all the endogenous peptides P is ended (Yes in Step S104), all variations of the target peptide sequence are stored in the target peptide sequence database 111.

For example, as shown in FIG. 2, an N-terminal side of the peptide sequence of the endogenous peptide P is extended, and a C-terminal side thereof is contracted, whereby a peptide sequence of a target peptide P1 can be generated. Moreover, the C-terminal side is extended, and the N-terminal side is contracted, whereby a peptide sequence of a target peptide P2 can be generated. Furthermore, both of the N-terminal side and C-terminal side of the peptide sequence of the endogenous peptide P are contracted, whereby a peptide sequence of a target peptide P3 can be generated. In addition, both of the N-terminal side and the C-terminal side are extended, whereby a peptide sequence of a target peptide P4 can be generated. However, as shown by P5 and P6 in FIG. 2, peptide sequences which do not share partial sequences with the endogenous peptide P are not generated as such target peptide sequences. Therefore, it is possible to suppress the search space to be smaller even in comparison with the case where the full-length sequence of the assignment protein in the known peptide is cleaved in a non-specific manner and peptide sequences are generated. Note that, when an isoform is present in the assignment protein, and a registered sequence is identical but the sequence to be extended is different, then the isoform is generated as a target peptide sequence of a different variation, and is stored in the target peptide sequence database 111.

### 3. PROCESSING BY MASS SPECTROMETRY UNIT AND PEAK LIST CREATION UNIT

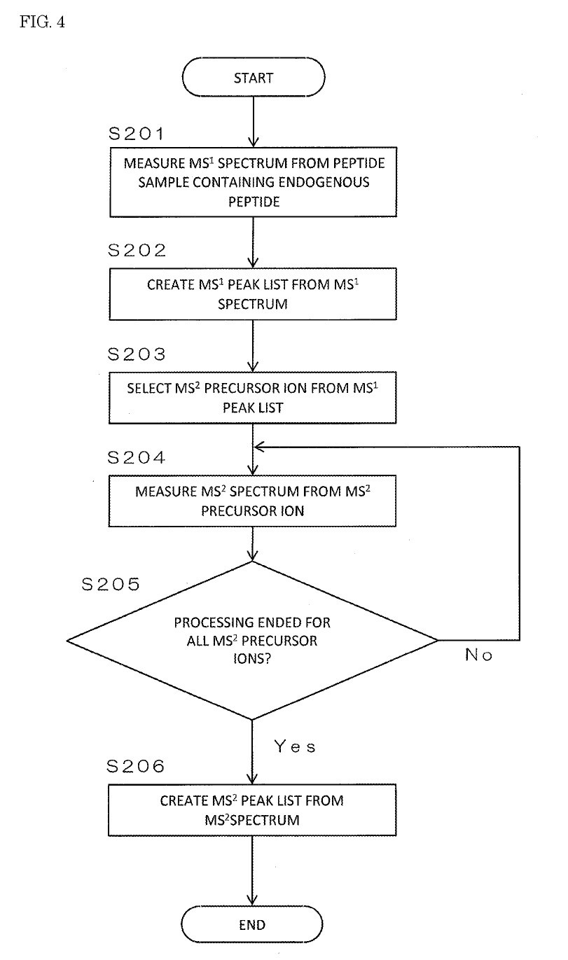

FIG. 4 is a flowchart showing processing by the mass spectrometry unit 12 and the peak list creation unit 13.

The mass spectrometry unit 12 ionizes a peptide sample containing the endogenous peptide, and measures an MS¹ spectrum by mass spectrometry of the ion (Step S201). At this time, the peak list creation unit 13 creates the MS¹ peak list by extracting peaks from the measured MS¹ spectrum (Step S202).

Thereafter, the mass spectrometry unit 12 selects a plurality of the MS² precursor ions to be measured targets of an MS² spectrum from the created MS¹ peak list by a predetermined method (Step S203), cleaves each MS² precursor ion and performs mass spectrometry to measure the MS² spectrum (Step S204). The processing in Step S204 is performed for all the MS² precursor ions. When the processing for all the MS² precursor ions is ended (Yes in Step S205), the peak list creation unit 13 extracts peaks from the measured MS² spectrum, thereby creating an MS² peak list (Step S206).

### 4. PROCESSING BY PEPTIDE ASSIGNMENT UNIT

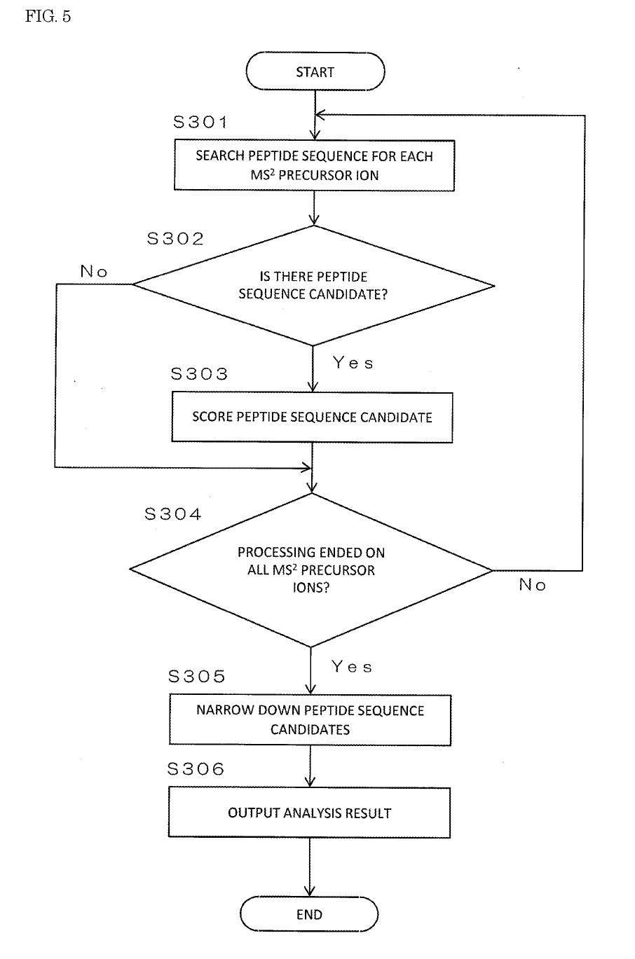

FIG. 5 is a flowchart showing processing by the peptide assignment unit 14.

For each MS² precursor ions in the MS¹ peak list, the sequence estimation part 141 searches the target peptide sequence database 111 for the peptide sequence that matches the MS² precursor ion mass within a predetermined mass tolerance range (Step S301). As a result, when at least one corresponding peptide sequence (peptide sequence candidate) is obtained (Yes in Step S302), the peptide sequence candidate is scored (Step S303).

When the peptide sequence candidate is scored, for example, a theoretical product ion mass of a main product ion (for example, y/b series ion) of the peptide sequence candidate is calculated, and for each product ion in the MS² peak list, a peptide sequence candidate that matches the theoretical product ion mass within a predetermined mass tolerance range is searched for. The main product ion means a product ion in which a site susceptible to cleavage is known beforehand. Since the site susceptible to cleavage is known beforehand, the theoretical product ion mass can be calculated.

Each of the peptide sequence candidates searched for is scored using matching peak intensities, peak numbers, and the like. As a score calculation method, various score calculation methods used in database search using protein databases can be adopted.

Processing in Steps S301 to S303 is performed for all the MS² precursor ions. When the processing for all the MS² precursor ions is ended (Yes in Step S304), the peptide sequence candidates are narrowed down based on a score of each peptide sequence candidate (Step S305). At this time, the peptide sequence candidates are uniquely narrowed down based on a significant difference between such scores, and peptide sequences thereof are output as an analysis result (Step S306). Note that if statistical indices cannot be calculated for reasons such as a small number of the peptide sequence candidates, then for example, processing up to ranking based on the scores may be performed, and the subsequent narrowing down may be left to a user.

### 5. FUNCTIONS AND EFFECTS

In the present embodiment, the database (target peptide sequence database 111) of the peptide sequences each containing one or more residues of the partial sequence of the endogenous peptide is generated based on the endogenous peptide having the known peptide sequence. The peptide sequence (target peptide sequence) in which the partial sequence is partially common to the endogenous peptide whose peptide sequence is known may be left as a peptide sequence of an unknown endogenous peptide in a mass spectrum that is not assigned by a conventional method.

Therefore, if the database (target peptide sequence database 111) of the target peptide sequences is generated, then the increase in the search space can be effectively prevented. Then, based on the target peptide sequence database 111 and the mass spectrum obtained by the mass spectrometry of the peptide sample in the mass spectrometry unit 12, the peptide sequence of the target peptide contained in the peptide sample is preferentially searched for, whereby more peptide sequences of the endogenous peptides can be determined with high sensitivity.

### 6. EXAMPLE

Based on 944 peptide sequences of the endogenous peptides contained in the aforementioned Mosaique DB and of the endogenous peptides assigned from the measurement data, 944,390 variations of the target peptide sequence composed of 7 to 80 residues in length were generated, and a target peptide database was created.

Peptide sequences were estimated by the sequence estimation part for MS² measurement data and MS³ measurement data of approximately 38 peaks (precursor ions: m/z=793 to 2943, a total of 70 spectra) measured from urine samples by the mass spectrometry unit. As a result, for 35 peaks (excluding those having precursor ion masses overlapping in the mass tolerance range and having different peptide sequences), peptide sequence candidates which match the target peptide sequence stored in the target peptide sequence database within the mass tolerance range were obtained, and approximately 50 (a total of over 1,800) peptide sequence candidates were obtained on average from each peak.

For the peptide sequence candidates estimated by the sequence estimation part, the theoretical masses of the y/b series ions were calculated, and the product ions were collated with a total of 70 MSⁿ spectra (n=2 or 3) obtained from peaks to be analyzed. Then, according to a score calculation method similar to X! Tandem which is a known search engine, scores were given as follows. However, a scoring method is not limited to this example, and various methods adopted in the database search method as a conventional method may be adopted.

Such scoring was carried out using following formulas (1) and (2).

$$[ Expression 1 ]$$

$$\begin{matrix}Score = \{ \begin{matrix}10 * {log}_{10} ( HyperScore ) & if HyperScore > 1 \\ 0.0 & otherwise \end{matrix} & ( 1 ) \\ HyperScore = ( {\sum }_{i}^{N} {I}_{i} ) * {n}_{b} ! * {n}_{y} ! \text{/} TIC & ( 2 )\end{matrix}$$

Here, Score is a score actually calculated from the peptide sequence candidate and the measurement data. Iᵢ is intensity of each of the peaks which matched as a result of the collation, N is a total number of the matched peaks, TIC is a total ion chromatogram of the MS² spectrum to be searched, n_{b} and n_{y} are numbers of b-series ions and y-series ions, which matched as a result of the product ion collation, and here, N=n_{b}+n_{y}. Based on a score distribution of the peptide sequence candidates, it is possible to provide indices and thresholds for selecting statistically significant sequences from the sequence candidates. For example, it is possible to set a discrimination threshold with a significance probability (p-value) or an expectation value (E-value), which is calculated from the score distribution, taken as an index. However, an index for discriminating the presence or absence of the significant difference is not limited to the index as mentioned above, and in the present example, it is possible to replace (reproduce) the discrimination based on the E-value by a discrimination method with a score difference between a first ranking candidate and a lower ranking candidate taken as a threshold.

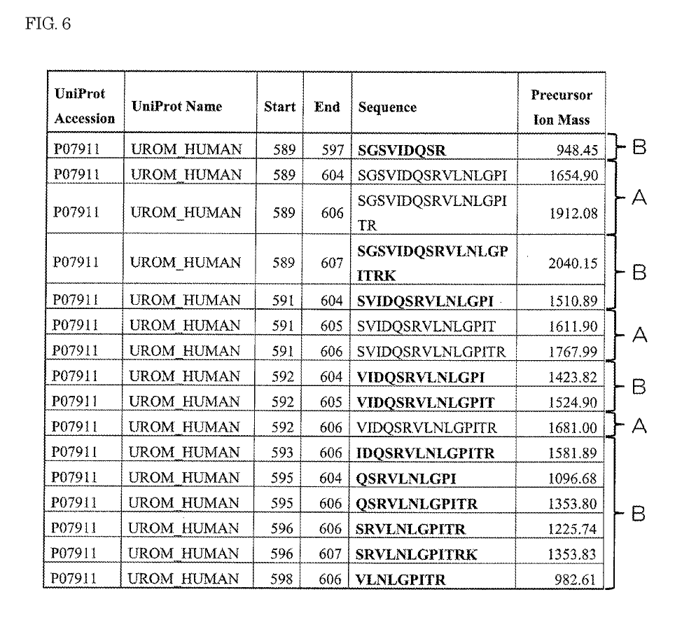

FIG. 6 is a table showing results of actually analyzing the MS² spectra obtained from the urine samples. “UniProt Accession” is a protein ID in UniProt as a protein database. “UniProt Name” is a name of a registered protein in UniProt. “Start” and “End” indicate positions of starting and ending residues of a peptide in a registered sequence in UniProt. “Sequence” is an amino acid sequence of an assigned urinary peptide. “Precursor Ion Mass” is a mass-to-charge ratio of monovalent peptide ions observed by mass spectrometry

Mascot or X! Tandem which is a protein database search method as a conventional method was used for 16 high-quality MS² spectra obtained from the urine samples for evaluation. In this case, only five peptide sequences (A in FIG. 6) were identified though identification thresholds were set to 1.0 and 0.1, respectively for the above methods, which were significantly relaxed as compared with thresholds used in proteome analysis, and false positive hits were allowed.

In contrast, as a result of the analysis according to the present invention, peptide sequence candidates were estimated from all of 16 high quality MS² spectra including not only the above five peptide sequences (A in FIG. 6) but also 11 peptide sequences (B in FIG. 6). Then, as a result of scoring by the product ion collation part, the score difference between the first ranking candidate and a second ranking candidate or less was 10 or more for any spectrum. Therefore, it was determined that the first ranking candidate was a significant estimation result. In addition, as a result of visual inspection, it was recognized that all of the estimation results were valid results.

### Second Embodiment

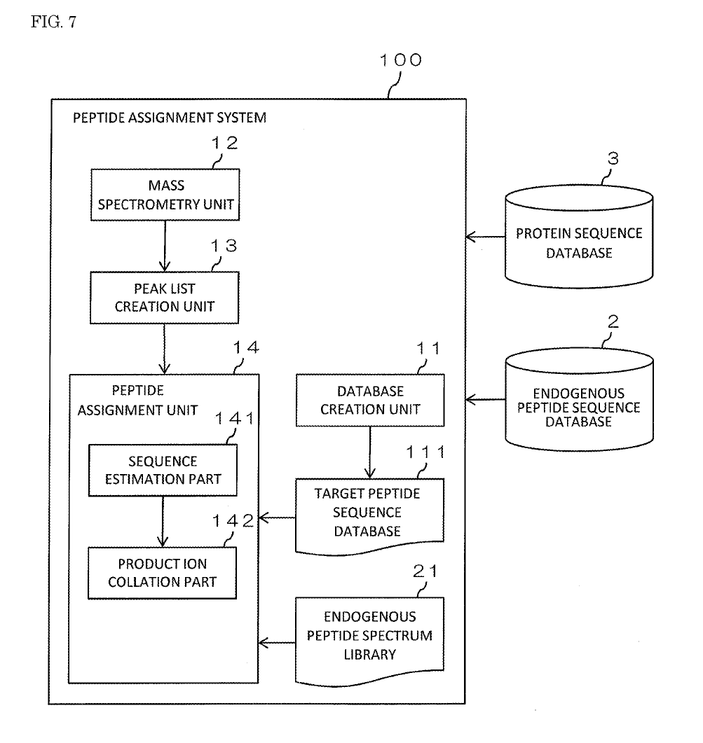

FIG. 7 is a block diagram showing a configuration example of a peptide assignment system 100 according to a second embodiment of the present invention.

In the first embodiment, the description has been given of the configuration in which the product ion collation part 142 calculates the theoretical product ion mass from the peptide sequence candidates when collating the main product ions. In contrast, in the second embodiment, the product ion collation part 142 calculates a similarity with MSⁿ measurement data (collation source data) to be analyzed using MSⁿ measurement data (collation destination data) of the endogenous peptide in which the peptide sequences are stored in the endogenous peptide sequence database 2 from which the peptide sequence candidates were created. Since other configurations are the same as those of the first embodiment, the same reference numerals are given in the drawings, and a description thereof is omitted.

The peptide assignment system 100 includes an endogenous peptide spectrum library 21. The endogenous peptide spectrum library 21 stores MSⁿ spectra obtained by the mass spectrometry for each of endogenous peptides in which the peptide sequences are stored in the endogenous peptide sequence database 2. Using the MSⁿ spectra stored in the endogenous peptide spectrum library 21, the product ion collation part 142 calculates the similarity with the MSⁿ spectra measured by the mass spectrometry unit 12. If Δm obtained by subtracting the precursor ion mass of the collation source data from the precursor ion mass of the collation destination data is larger than the mass tolerance, then a collation result with a peak obtained by subtracting Δm from the product ion mass of the collation source data is also used for calculating the similarity. Moreover, if Δm is larger than a mass Δn of a sequence (having one or more amino acids) at either end of the peptide sequence of the collation source, then collation result with a peak obtained by subtracting Δn from the product ion mass of the collation source may be used for calculating the similarity.

For calculating the similarity, various methods used in the known spectrum library search method can be used (for example, Stein, S. E. & Scott, D. R.: Optimization and Testing of Mass Spectral Library Search Algorithms for Compound Identification. JASMS, 5, 859-866 (1994)). In this case, for example, the collation source data and the collation destination data may be collated with each other, and normalized products of peak intensities for ion peaks which coincide with each other within the mass tolerance range may be used as the similarity.

In the case of endogenous peptides, there are cases where the endogenous peptides are cleaved at unexpected sites. Therefore, in the case of the configuration in which the theoretical product ion mass is calculated as in the first embodiment, there is a possibility that the scoring cannot be performed theoretically. In contrast, in the second embodiment, the actual MSⁿ measurement data of the endogenous peptides in which the peptide sequences are stored in the endogenous peptide sequence database 2 is used, and therefore, there are cases where the peptide sequence can be determined with higher sensitivity.

### DESCRIPTION OF REFERENCE SIGNS

1 peptide assignment system
2 endogenous peptide sequence database
11 database creation unit
12 mass spectrometry unit
13 peak list creation unit
14 peptide assignment unit
21 endogenous peptide spectrum library
100 peptide assignment system
111 target peptide sequence database
141 sequence estimation part
142 product ion collation part

1. Apeptide assignment method comprising:
a database creation step of, based on an endogenous peptide whose peptide sequence is known among endogenous peptides produced in vivo and on a full-length sequence of a precursor protein of the endogenous peptide, generating a peptide sequence as a target peptide sequence, the peptide sequence containing one or more residues of a partial sequence of the endogenous peptide, and creating a target peptide sequence database including a plurality of the target peptide sequences;
a mass spectrometry step of performing mass spectrometry on a peptide sample; and
a peptide assignment step of determining a peptide sequence of an endogenous peptide contained in the peptide sample based on the plurality of target peptide sequences generated by the database creation step and on a mass spectrum obtained by the mass spectrometry step.

2. Apeptide assignment system comprising:
a database creation unit that, based on an endogenous peptide whose peptide sequence is known among endogenous peptides produced in vivo and on a full-length sequence of a precursor protein of the endogenous peptide, generates a peptide sequence as a target peptide sequence, the peptide sequence containing one or more residues of a partial sequence of the endogenous peptide, and creates a target peptide sequence database including a plurality of the target peptide sequences;
a mass spectrometry unit that performs mass spectrometry on a peptide sample; and
a peptide assignment unit that determines a peptide sequence of an endogenous peptide contained in the peptide sample based on the plurality of target peptide sequences generated by the database creation unit and on a mass spectrum obtained by the mass spectrometry unit.
